(19) United States
(12) Patent Application Publication
(10) Pub. No.: US 2016/0083397 A1
(43) Pub. Date: Mar. 24, 2016

(54) PARTHENOLIDE DERIVATIVES AND THEIR MODULATION OF PROCESSES CONTROLLED BY REGULATED TRANSLATION

(71) Applicants: Narsimha Reddy PENTHALA, Little Rock, AR (US); Peter CROOKS, Little Rock, AR (US); Venumadhav JANGANATI, Little Rock, AR (US); THE BOARD OF TRUSTEES OF THE UNIVERSITY OF ARKANSAS, Little Rock, AR (US)

(72) Inventors: Narsimha Reddy Penthala, Little Rock, AR (US); Peter Crooks, Little Rock, AR (US); Venumadhav Janganati, Little Rock, AR (US)

(21) Appl. No.: 14/785,183

(22) PCT Filed: Apr. 18, 2014
(86) PCT No.: PCT/US14/34605

§ 371 Date: Oct. 16, 2015

Related U.S. Application Data
(60) Provisional application No. 61/814,020, filed on Apr. 19, 2013.
(60) Provisional application No. 61/866,872, filed on Aug. 16, 2013.
(60) Provisional application No. 61/871,657, filed on Aug. 29, 2013.

(51) Int. Cl. C07D 493/08

(52) U.S. Cl. CPC C07D 493/08

(57) ABSTRACT

The present invention provides parthenolide derivatives. In particular, the present invention provides parthenolide derivatives that modulate processes controlled by regulated mRNA translation and have anticancer activity.

### CROSS-REFERENCE TO RELATED APPLICATIONS

This application claims the benefit of PCT Application PCT/US2014/034605, filed Apr. 18, 2014, which claims the benefit of U.S. Provisional Patent Application No. 61/814,020, filed Apr. 19, 2013, U.S. Provisional Patent Application No. 61/866,872, filed Aug. 16, 2013, and U.S. Provisional Patent Application No. 61/871,657 filed Aug. 29, 2013, which are each hereby incorporated by reference in their entirety.

### GOVERNMENTAL RIGHTS

This invention was made with government support under CA158275I awarded by the National Institutes of Health. The government has certain rights in the invention.

### FIELD

The present disclosure relates to parthenolide derivatives. In particular, the present disclosure relates to parthenolide derivatives that modulate cell cycle progression, cell growth control, cell survival, and other processes controlled by regulated mRNA translation.

### BACKGROUND

Parthenolide (PTL), a naturally occurring sesquiterpene lactone isolated from Tanacetum parthenium (feverfew), is used for the treatment of fever, migraine headaches, rheumatoid arthritis, and also as an anti-inflammatory agent. In recent years the PTL molecule and several structurally related sesquiterpene lactone analogs have been extensively studied because of their potent anticancer and cytotoxic properties. PTL has been shown to target NF-κB, Stat3, HDAC, SERCA, and COX-2 in cancer cells. A related analog, melampomagnolide B (MMB), has been reported to have antileukemic activity.

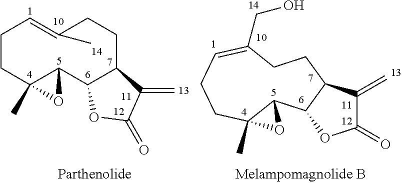

Despite the promising in vitro activity of these compounds, their further development as therapeutic agents is limited by their low water-solubility and poor bioavailability. What is needed, therefore, are parthenolide derivatives that have increased water solubility and robust cytotoxic anticancer properties.

### SUMMARY

Among the various aspects of the present disclosure is the provision of a method for modulating a process controlled by regulated mRNA translation. The method comprises contacting a cell with an effective amount of a compound comprising Formula (I) or a pharmaceutically acceptable salt thereof, whereby the process controlled by regulated mRNA translation is attenuated or activated. The compound comprising Formula (I) has the following structure:

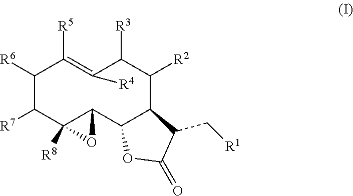

wherein:
R¹ is hydrogen, hydrocarbyl, or substituted hydrocarbyl;
R², R³, R⁶, and R⁷ are independently hydrogen, hydroxyl, keto, halo, amine, amide, nitro, phospho, cyano, thiol, hydrocarbyl, or substituted hydrocarbyl;
R⁴, R⁵, and R⁸ are independently hydrogen, hydroxyl, halo, amine, amide, nitro, phospho, thiol, hydrocarbyl, or substituted hydrocarbyl; and
----- is a double or single bond.

Another aspect of the present disclosure provides a compound comprising Formula (II) or a pharmaceutically acceptable salt thereof:

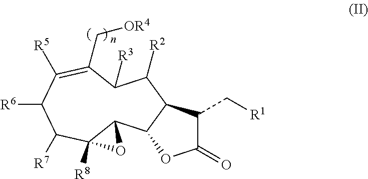

wherein:
R¹ is hydrocarbyl or substituted hydrocarbyl;
R², R³, R⁶, and R⁷ are independently hydrogen, hydroxyl, keto, halo, amine, amide, nitro, phospho, cyano, thiol, hydrocarbyl, or substituted hydrocarbyl;
R⁴ is hydrogen, hydrocarbyl, or substituted hydrocarbyl;
R⁵ and R⁸ are independently hydrogen, hydroxyl, halo, amine, amide, nitro, phospho, thiol, hydrocarbyl, or substituted hydrocarbyl;
n is an integer from 1 to 4; and
----- is a double or single bond.

A further aspect of the present disclosure encompasses a compound comprising Formula (III) or a pharmaceutically acceptable salt thereof:

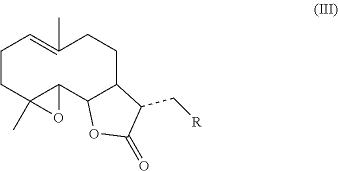

wherein:
----- is a double or single bond; and
R is —NR⁹R¹⁰, in which R⁹ is hydrogen and R¹⁰ is heterocyclic or substituted heterocyclic, or together R⁹ and R¹⁰ form heterocyclic or substituted heterocyclic when ----- is a single bond; or
R is carbocyclic, substituted carbocyclic, heterocyclic, or substituted heterocyclic when ----- is a double bond.

Still another aspect of the present disclosure provides a method for inhibiting cancer cell growth, the method comprising administering to a subject in need thereof an effective amount of a compound comprising Formula (III), Formula (II), or a pharmaceutically acceptable salt of either:

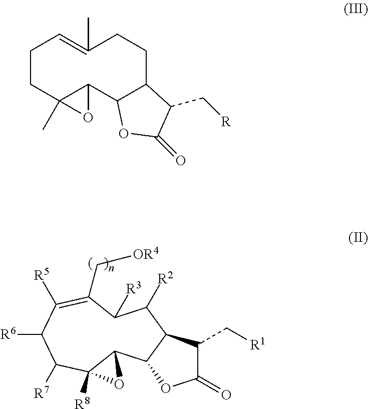

wherein:
----- is a double or single bond; R is —NR⁹R¹⁰, in which R⁹ is hydrogen and R¹⁰ is heterocyclic or substituted heterocyclic, or together R⁹ and R¹⁰ form heterocyclic or substituted heterocyclic when ----- is a single bond; or
R is carbocylic, substituted carbocyclic, heterocyclic, or substituted heterocyclic when ----- is a double bond;
R¹ is hydrocarbyl or substituted hydrocarbyl;
R², R³, R⁶, and R⁷ are independently hydrogen, hydroxyl, keto, halo, amine, amide, nitro, cyano, phospho, thiol, hydrocarbyl, or substituted hydrocarbyl;
R⁴ is hydrogen, hydrocarbyl, or substituted hydrocarbyl;
R⁵ and R⁸ are independently hydrogen, hydroxyl, halo, amine, amide, nitro, cyano, phospho, thiol, hydrocarbyl, or substituted hydrocarbyl; and
n is an integer from 1 to 4.

Other aspects and iterations of the disclosure are described in more detail below.

### BRIEF DESCRIPTION OF THE DRAWINGS

The application file contains at least one photograph executed in color. Copies of this patent application publication with color photographs will be provided by the Office upon request and payment of the necessary fee.

FIG. 1A and FIG. 1B show inhibition of breast cancer cell self-renewal by parthenolide derivatives. Plotted is the percent of mammosphere-forming units (MFUs) at day 14 (P2) per cells plated (at day 7, P1) for cells treated with each of the indicated compounds (until day 3). FIG. 1A shows MCF-7 cells. FIG. 1B shows MDA-MB-231 cells. Values with different letters (a, b, c) differed at P<0.05. N=3 experiments, carried out in quadruplicate.

FIG. 2A and FIG. 2B present inhibition of neural cancer cell self-renewal by parthenolide derivatives. Plotted is the percent of sphere formation relative to DMSO-treated cells for the indicated compounds at the indicated doses. FIG. 2A shows SHSY5Y 7 cells. FIG. 2B presents U87 cells. * P<0.05; ** P<0.01; *** P<0.001 by ANOVA. N=3 experiments, carried out in quadruplicate.

### DETAILED DESCRIPTION

Provided herein are derivatives of parthenolide that modulate the activity of processes involved in cell growth and cell survival. In particular, aminoparthenolide derivations are provided that have improved water solubility relative to the parent compound. Moreover, the present disclosure reveals that parthenolide derivatives modulate key cellular processes controlled by regulated mRNA translation. Regulated mRNA translation is involved in the control of critical cellular processes such as cell cycle progression, cell growth, cell survival, and signal transduction.

### (I) Compounds Comprising Formula (II)

One aspect of the present disclosure provides a compound comprising Formula (II) or a pharmaceutically acceptable salt thereof:

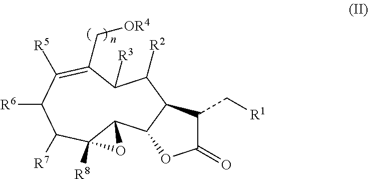

wherein:
R¹ is hydrocarbyl or substituted hydrocarbyl;
R², R³, R⁶, and R⁷ are independently hydrogen, hydroxyl, keto, halo, amine, amide, nitro, phospho, cyano, thiol, hydrocarbyl, or substituted hydrocarbyl;
R⁴ is hydrogen, hydrocarbyl, or substituted hydrocarbyl;
R⁵ and R⁸ are independently hydrogen, hydroxyl, halo, amine, amide, nitro, phospho, thiol, hydrocarbyl, or substituted hydrocarbyl;
n is an integer from 1 to 4; and
----- is a double or single bond.

In some embodiments, R¹ may be alkyl, substituted alkyl, alkenyl, substituted alkenyl, alkynyl, substituted alkynyl, amine, carbocyclic, substituted carbocyclic, heterocyclic, or substituted heterocyclic. The terms “carbocyclic” and “heterocyclic” include both saturated and unsaturated rings or ring systems. (Thus, the term “aryl” includes both carbocyclic and heterocyclic.) In certain embodiments, R¹ may be an amine, which may be linear or cyclic. As an example, R¹ may be —NR⁹R¹⁰, in which R⁹ is hydrogen and R¹⁰ is alkyl, substituted alkyl, alkenyl, substituted alkenyl, alkynyl, substituted alkynyl, carbocyclic, substituted carbocyclic, heterocyclic, or substituted heterocyclic. Alternatively, R¹ may be —NR⁹R¹⁰, in which together R⁹ and R¹⁰ form a ring (i.e., 5- or 6-membered) or ring system (i.e., bicyclic, tricyclic, etc.) chosen from carbocyclic, substituted carbocyclic, heterocyclic, substituted heterocyclic or combinations thereof.

In certain embodiments, R⁴ may be hydrogen, alkyl, substituted alkyl, alkenyl, substituted alkenyl, alkynyl, substituted alkynyl, aryl, or substituted aryl. In some embodiments, R⁴ may be hydrogen or C₁-C₄ alkyl. In various embodiments, n may be 1.

In other embodiments, R², R³, R⁶, and R⁷ independently may be hydrogen, hydroxyl, alkyoxy, keto, halo, amine, alkyl, substituted alkyl, alkenyl, substituted alkenyl, alkynyl, substituted alkynyl, aryl, or substituted aryl; and R⁵ and R⁸ independently may be hydrogen, hydroxyl, alkyoxy, halo, amine, alkyl, substituted alkyl, alkenyl, substituted alkenyl, alkynyl, substituted alkynyl, aryl, or substituted aryl. In one embodiment, each of R², R³, R⁵, R⁶, and R⁷ may be hydrogen. In other embodiments, R⁸ may be alkyl. In one instance, R⁸ may be methyl.

The compounds comprising Formula (II) can exist in tautomeric, geometric, or stereoisomeric forms. For example, the carbons at positions 4, 5, 6, 7, and 11 may be stereogenic (or chiral). At each chiral center, the stereochemistry at the carbon atom is independently R or S. In embodiments in which C-4, C-5, C-6, and C-7 are chiral, the configuration of C-4, C-5, C-6, and C-7 may be RRRR, RRRS, RRSR, RSRR, SRRR, RRSS, RSSR, SSRR, SRRS, SRSR, RSRS, RSSS, SRSS, SSRS, SSSR, or SSSS. In embodiments in which the optional single bond is present, C-11 is also chiral and its configuration may be R or S. In embodiments in which the optional double bond is present, each compound may have a cis (E) or trans (2) geometric form.

In some embodiments, the compound comprises Formula (IIa):

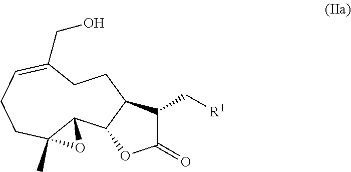

wherein:
R¹ is —NR⁹R¹⁰, in which R⁹ is hydrogen and R¹⁰ is alkyl, substituted alkyl, alkenyl, substituted alkenyl, alkynyl, substituted alkynyl, carbocyclic, substituted carbocyclic, heterocyclic, or substituted heterocyclic, or together R⁹ and R¹⁰ form a ring or ring system chosen from carbocyclic, substituted carbocyclic, heterocyclic, or substituted heterocyclic.

In various iterations of this embodiment, R¹ may be chosen from:

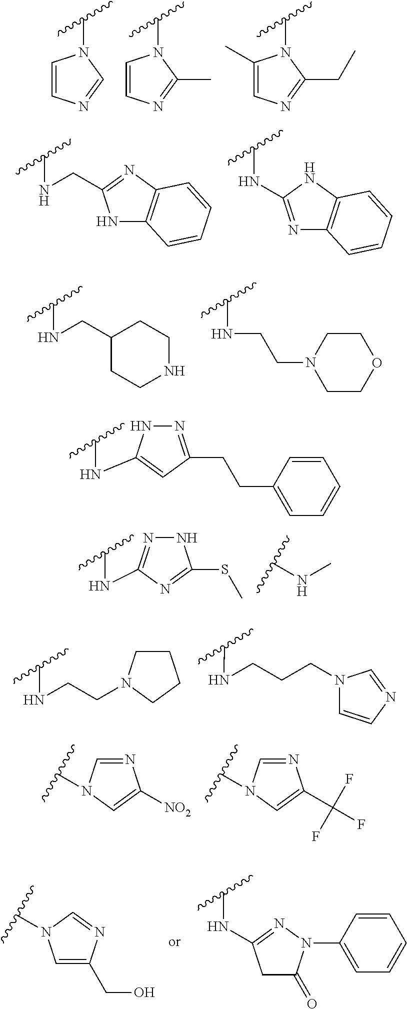

In further embodiments, the compound comprises Formula (IIb):

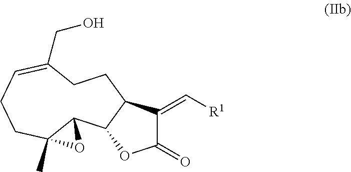

wherein R¹ is carbocyclic, substituted carbocyclic, heterocyclic, or substituted heterocyclic.

In some iterations of this embodiments, R¹ may be chosen from:

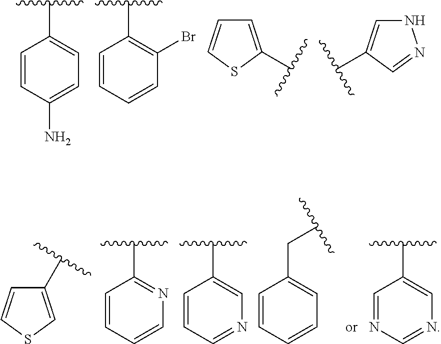

In one embodiment, the compound comprising Formula (IIb) has a cis (E) geometry about the double bond to which R¹ is attached. In another embodiment, the compound comprising Formula (IIb) has a trans (Z) about the double bond to which R¹ is attached.

In one aspect of the disclosure, the compounds comprising Formula (II) are characterized by improved solubility. In some aspects, the solubility of the compound comprising Formula (II) may be greater than about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, or about 200 μM. In one aspect, the solubility of the compound comprising Formula (II) is about 100 μM.

### (II) Compounds Comprising Formula (III)

Another aspect of the present disclosure encompasses a compound comprising Formula (III):

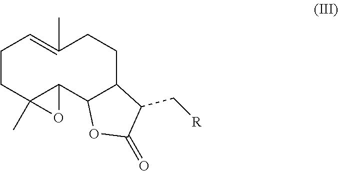

wherein:
----- is a double or single bond; and
R is —NR⁹R¹⁰, in which R⁹ is hydrogen and R¹⁰ is heterocyclic or substituted heterocyclic, or together R⁹ and R¹⁰ form heterocyclic or substituted heterocyclic when ----- is a single bond; or
R is carbocyclic, substituted carbocyclic, heterocyclic, or substituted heterocyclic when ----- is a double bond.

The compounds comprising Formula (III) can exist in tautomeric, geometric, or stereoisomeric forms. For example, the carbons at positions 4, 5, 6, 7, and 11 may be stereogenic (or chiral). At each chiral center, the stereochemistry at the carbon atom is independently R or S. In embodiments in which C-4, C-5, C-6, and C-7 are chiral, the configuration of C-4, C-5, C-6, and C-7 may be RRRR, RRRS, RRSR, RSRR, SRRR, RRSS, RSSR, SSRR, SRRS, SRSR, RSRS, RSSS, SRSS, SSRS, SSSR, or SSSS. In embodiments in which the optional single bond is present, C-11 is also chiral and its configuration may be R or S. In embodiments in which the optional double bond is present, each compound may have a cis (E) or trans (2) geometric form.

In some embodiments, the compound comprises Formula (IIIa):

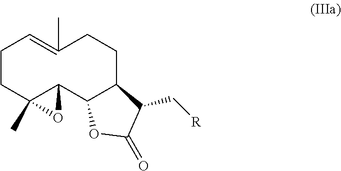

wherein R is chosen from:

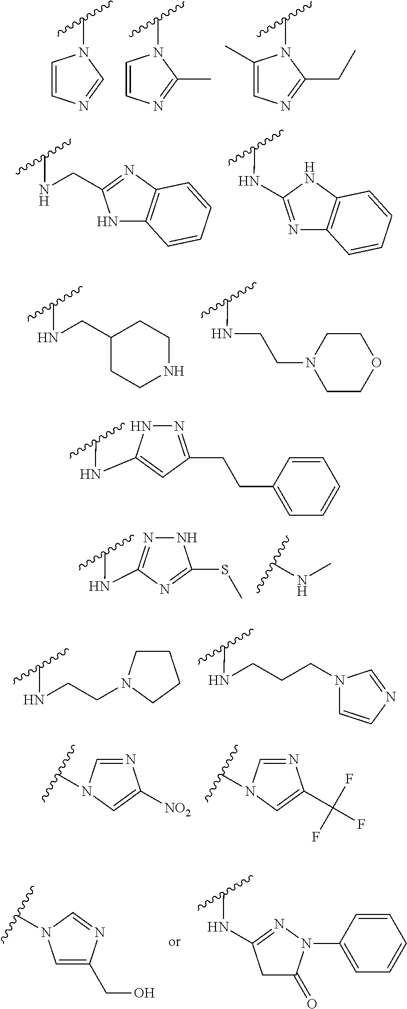

In other embodiments, the compound comprises Formula (IIIb):

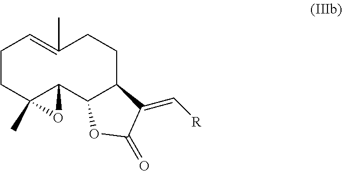

wherein R is chosen from:

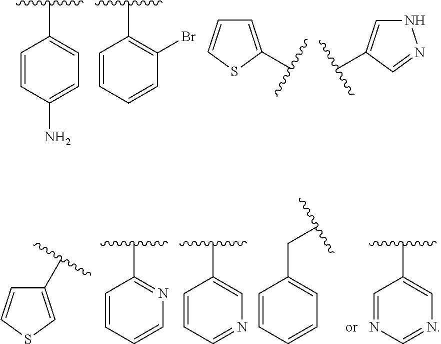

In one embodiment, the compound comprising Formula (IIIb) has a cis (E) geometry. In another embodiment, the compound comprising Formula (IIIb) has a trans (2) geometry.

In one aspect of the disclosure, the compounds comprising Formula (III) are characterized by improved solubility. In some aspects, the solubility of the compound comprising Formula (III) may be greater than about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, or about 200 μM. In one aspect, the solubility of the compound comprising Formula (III) is about 100 μM.

The compounds comprising Formulas (II), (IIa), (IIb), (III), (IIIa), and (IIIb) disclosed herein may be in the form of free bases or pharmaceutically acceptable salts thereof. The term “pharmaceutically acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds disclosed herein may be prepared from inorganic acids or organic acids. Non-limiting examples of suitable inorganic acids include hydrochloric, hydrobromic, hydroiodic, nitric, carbonic, sulfuric, perchloric, and phosphoric acid. Appropriate organic acids may be selected from aliphatic, cycloaliphatic, aromatic, araliphatic, heterocyclic, carboxylic and sulfonic classes of organic acids, examples of which are formic, acetic, oxalic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically acceptable base addition salts of compounds disclosed herein include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine), and procaine. All of these salts may be prepared by conventional means from the corresponding compound by reacting, for example, the appropriate acid or base with the any of the compounds disclosed herein.

The compounds disclosed herein may be prepared by a variety of methods. For example, the compounds comprising Formulas (IIa) and (IIIa) may be prepared by contacting parthenolide with an appropriate amine, in the presence of a suitable solvent (e.g., methanol, chloroform, etc.), and at a temperature ranging from about 20° C. to reflux of the solvent. The compounds comprising Formulas (IIb) and (IIIb) may be prepared by contacting parthenolide with a halogenated aromatic or heteroaromatic compound and a palladium acetate catalyst in the presence of a suitable solvent (e.g., dimethyl formamide), and at a temperature ranging from about 70° C. to about 100° C. The parthenolide derivatives may be converted to melamponmagnolide B analogs by contact with selenium dioxide and t-butyl hydroperoxide.

### (III) Compositions

The present disclosure also provides pharmaceutical compositions. A composition comprises a compound comprising Formula (II), (IIa), (IIb), (III), (IIIa), or (IIIb), which are detailed above in section (I) and (II), respectively, as an active ingredient and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may include at least one diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may include a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may comprise a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may include a pH modifier. By way of non-limiting example, the pH modifying agent may be sodium carbonate, sodium bicarbonate, sodium citrate, citric acid, or phosphoric acid.

In a further embodiment, the excipient may include a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may include a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may also include a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may include a lubricant. Non-limiting examples of suitable lubricants include minerals such as talc or silica; and fats such as vegetable stearin, magnesium stearate or stearic acid.

In yet another embodiment, the excipient may comprise a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may comprise a flavoring agent. Flavoring agents may be chosen from synthetic flavor oils and flavoring aromatics and/or natural oils, extracts from plants, leaves, flowers, fruits, and combinations thereof.

In still a further embodiment, the excipient may include a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, caplets, pills, powders, pellets, and granules. In such solid dosage forms, the active ingredient is ordinarily combined with one or more pharmaceutically acceptable excipient, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

For topical (e.g., transdermal or transmucosal) administration, penetrants appropriate to the barrier to be permeated are generally included in the preparation. Transmucosal administration may be accomplished through the use of nasal sprays, aerosol sprays, tablets, or suppositories, and transdermal administration may be via ointments, salves, gels, patches, or creams as generally known in the art.

(IV) Methods for Modulating a Process Controlled by Regulated mRNA Translation

A further aspect of the present disclosure provides methods for modulating a process controlled by regulated mRNA translation. The method comprises contacting a cell with an effective amount of a compound comprising Formula (I) or a pharmaceutically acceptable salt thereof, such that the process controlled by regulated mRNA translation is attenuated or activated. The compound comprising Formula (I):

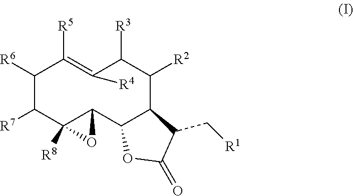

wherein:
R¹ is hydrogen, hydrocarbyl, or substituted hydrocarbyl;
R², R³, R⁶, and R⁷ are independently hydrogen, hydroxyl, keto, halo, amine, amide, nitro, phospho, cyano, thiol, hydrocarbyl, or substituted hydrocarbyl;
R⁴, R⁵, and R⁸ are independently hydrogen, hydroxyl, halo, amine, amide, nitro, phospho, thiol, hydrocarbyl, or substituted hydrocarbyl; and
----- is a double or single bond.

In some embodiments, R¹ may be hydrogen, alkyl, substituted alkyl, alkenyl, substituted alkenyl, alkynyl, substituted alkynyl, amine, carbocyclic, substituted carbocyclic, heterocyclic, or substituted heterocyclic. In one embodiment, R¹ is hydrogen. In another embodiment R¹ is an amine; which may be linear or cyclic. For example, R¹ may be —NR⁹R¹⁰, in which R⁹ is hydrogen and R¹⁰ is alkyl, substituted alkyl, alkenyl, substituted alkenyl, alkynyl, substituted alkynyl, carbocyclic, substituted carbocyclic, heterocyclic, or substituted heterocyclic. Alternatively, R¹ may be —NR⁹R¹⁰, in which together R⁹ and R¹⁰ form a ring (i.e., 5- or 6-membered) or ring system (i.e., bicyclic, tricyclic, etc.) chosen from carbocyclic, substituted carbocyclic, heterocyclic, substituted heterocyclic or combinations thereof.

In other embodiments, R², R³, R⁶, and R⁷ independently may be hydrogen, hydroxyl, alkyoxy, keto, halo, amine, alkyl, substituted alkyl, alkenyl, substituted alkenyl, alkynyl, substituted alkynyl, aryl, or substituted aryl; and R⁴, R⁵, and R⁸ independently may be hydrogen, hydroxyl, alkyoxy, halo, amine, alkyl, substituted alkyl, alkenyl, substituted alkenyl, alkynyl, substituted alkynyl, aryl, or substituted aryl. In one embodiment, each of R², R³, R⁵, R⁶, and R⁷ may be hydrogen. In another embodiment, R⁴ may be alkyl, alkyl alcohol, ether, or ester. For example, R⁴ may be methyl or —CH₂OH. In other embodiments, R⁸ may be alkyl. In one instance, R⁸ may be methyl.

In various embodiments, the compound comprising Formula (I) may be a compound comprising Formula (II) or Formula (III), which are detailed above in sections (I) and (II), respectively.

The process comprises contacting a cell with an effective amount of the compound comprising Formula (I). An “effective” amount refers to the dose of the compound that affects the process (either positively or negatively). The amount to be used depends to some extent on the lipophilicity of the specific compound selected, since it is expected that this property of the compounds will cause it to partition efficiently into cells. The precise amount to be used can be determined by the skilled practitioner in view of desired dosages and side effects of the compound.

The type of cell that is contacted with the compound can and will vary. In some embodiments, the cell may be in vitro. The cell may be a primary cell or a cultured cell line cell. The cell line may be a human cell line or a mammalian cell line. The cell line may be a cancer cell line. In other embodiments, the cell may be an oocyte or an embryo. In one iteration, the oocyte or embryo may be amphibian. In another iteration, the oocycte or embryo may be frog (e.g., a Xenopus species).

In other embodiments, the cell may be in vivo; i.e., the cell may be disposed in a subject. In such embodiments, the cell is contacted with the compound comprising Formula (I) by administering the compound comprising Formula (I) to the subject. The compound comprising Formula (I) may be administered to the subject orally, parenterally, or topically. In some embodiments, the subject may be a human. In other embodiments, the subject may be a non-human animal. Non-limiting examples of non-human animals include companion animals (e.g., cats, dogs, horses, rabbits, gerbils), agricultural animals (e.g., cows, pigs, sheep, goats, fowl), research animals (e.g., rats, mice, rabbits, primates), and zoo animals (e.g., lions, tiger, elephants, and the like). In certain embodiments, the cell disposed in the subject may be a cancer cell or a tumor cell. The cancer may be primary or metastatic; the tumor may be malignant or benign. The cancer may be early stage or late stage. Non-limiting cancers include bladder cancer, bone cancer, brain cancer, breast cancer, cervical cancer, colorectal cancer, duodenal cancer, endometrial cancer, esophageal cancer, eye cancer, gallbladder cancer, germ cell cancer, kidney cancer, larynx cancer, leukemia, liver cancer, lymphoma, lung cancer, melanoma, mouth/throat cancer, ovarian cancer, pancreatic cancer, prostate cancer, skin cancer, testicular cancer, thyroid cancer, and vaginal cancer.

Regulated mRNA translation refers to a process in which protein synthesis is controlled by regulating the accessibility of mRNA transcripts. For example, translation can be regulated by a sequence-specific mRNA translational control protein, which binds to a specific mRNA in a sequence-specific manner and prevents translation of the message. Upon receipt of the appropriate signal, the sequence-specific mRNA translational control protein releases the specific mRNA, which then can be translated into protein. Alternatively, the sequence-specific mRNA translational control protein remains associated with the specific mRNA, but activity is altered to allow translation of the mRNA into protein. In one embodiment, the sequence-specific mRNA translational control protein is a Pumilio protein. In another embodiment, the sequence-specific mRNA translational control protein is a Musashi protein. In still another embodiment, the sequence-specific mRNA translational control protein is a cytoplasmic polyadenylation element binding (CPEB) protein. In another example, translation can be regulated by a sequence-specific mRNA translational control protein, which binds to a specific mRNA in a sequence-specific manner and promotes translation of the message. Upon receipt of the appropriate signal, the sequence-specific mRNA translational control protein activity is altered to prevent translation of the specific mRNA. In one embodiment, the sequence-specific mRNA translational control protein is a Musashi protein. In another embodiment, the sequence-specific mRNA translational control protein is a cytoplasmic polyadenylation element binding (CPEB) protein.

The process controlled by regulated mRNA translation may be cell cycle progression, cell growth control, cell division control (e.g., timing and/or symmetry of division), cell survival control, regulated cell death, nuclear signaling, DNA fragmentation, signal transduction, cell fate determination, cell differentiation, cell-cell interactions, cell-cell signaling, cell-cell contacts, cell adhesion, proteasome signaling, protein stability, or stem cell self-renewal. In some embodiments, the process controlled by regulated mRNA translation may be attenuated. For example, cell cycle progression or cell growth may be slowed or inhibited. In other embodiments, the process controlled by regulated mRNA translation may be activated. For example, a signal transduction process may be activated. The process controlled by regulated mRNA translation may be attenuated or activated about 0.5-fold, about 1-fold, about 2-fold, about 5-fold, about 10-fold, or more than 10-fold.

### (V) Methods for Inhibiting Cancer Cell Growth

A further aspect of the present disclosure provides a method for inhibiting growth of a cancer cell. The method comprises contacting the cell with an effective amount of a compound comprising Formula (III), Formula (II), or a pharmaceutically acceptable salt of either:

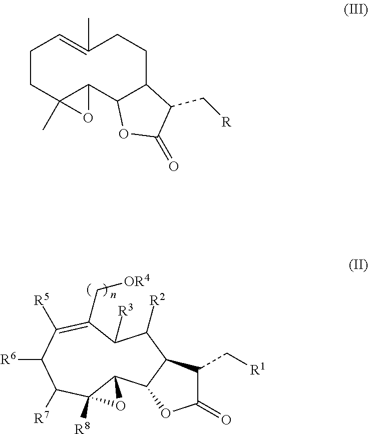

wherein:
----- is a double or single bond;
R is —NR⁹R¹⁰, in which R⁹ is hydrogen and R¹⁰ is heterocyclic or substituted heterocyclic, or together R⁹ and R¹⁰ form heterocyclic or substituted heterocyclic when ----- is a single bond; or
R is carbocylic, substituted carbocyclic, heterocyclic, or substituted heterocyclic when ----- is a double bond;
R¹ is hydrocarbyl or substituted hydrocarbyl;
R², R³, R⁶, and R⁷ are independently hydrogen, hydroxyl, keto, halo, amine, amide, nitro, cyano, phospho, thiol, hydrocarbyl, or substituted hydrocarbyl;
R⁴ is hydrogen, hydrocarbyl, or substituted hydrocarbyl;
R⁵ and R⁸ are independently hydrogen, hydroxyl, halo, amine, amide, nitro, cyano, phospho, thiol, hydrocarbyl, or substituted hydrocarbyl; and
n is an integer from 1 to 4.

Compounds comprising Formula (III) are detailed above in section (II). Compounds comprising Formula (II) are detailed above in section (I). In some embodiments, the compound comprising Formulas (II) or (III) is administered as part of a composition, examples of which are detailed above in section (III).

The method comprises contacting the cancer cell with an effective amount of one of the compound listed above. The type of cell that is contacted with the compound can and will vary. In some embodiments, the cancer cell may be in vitro. The cancer cell may be a primary cancer cell or a cultured cancer cell line cell. The cancer cell line may be a human cancer cell line or a mammalian cancer cell line. Examples of suitable cancer cell lines are listed below in Table 1. The in vitro cancer cell may be contacted with the compound comprising Formulas (II) or (III) continuously, for a short period of time, intermittently, or any of a variety of regimes.

In other embodiments, the cancer cell may be in vivo; i.e., the cancer cell may be disposed in a subject. In some embodiments, the subject may be a human. In other embodiments, the subject may be a non-human animal, examples of which are listed above in section (IV). In such embodiments, the cancer cell is contacted with the compound by administering the compound comprising Formulas (II) or (III) to the subject. The compound may be administered orally (as a solid or a liquid), parenterally (which includes intramuscular, intravenous, intradermal, intraperitoneal, and subcutaneous), or topically (which includes transmucosal and transdermal). As detailed above, an effective amount of the compound can be determined by a skilled practitioner. The compound comprising Formula (II) or (III) may be administered once or repeatedly to the subject. Repeated administrations may be at regular intervals of 2 hours, 6 hours, 12 hours, 24 hours, 2 days, 5 days, 7 days, 30 days, and so forth.

Following contact with the compound, the growth of the cancer cell is inhibited. In some embodiments, cancer cell growth may be inhibited about 0.5-fold, about 1-fold, about 2-fold, about 5-fold, about 10-fold, or more than 10-fold. In other embodiments, cancer cell growth may be inhibited to such a degree that the cell undergoes cell death (via apoptosis or necrosis).

In certain embodiments, the method may further comprise administering at least one chemotherapeutic agent and/or a radiotherapeutic agent. The chemotherapeutic agent and/or radiotherapeutic agent may be administered concurrently or sequentially.

The chemotherapeutic agent may be an alkylating agent, an anti-metabolite, an anti-tumor antibiotic, an anti-cytoskeletal agent, a topoisomerase inhibitor, an anti-hormonal agent, a targeted therapeutic agent, or a combination thereof. Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lomustine (CCNU), mechlorethamine, mechlorethamine oxide hydrochloride, melphalan, meturedopa, nimustine, novembichin, phenesterine, piposulfan, prednimustine, ranimustine; temozolomide, thiotepa, triethylenemelamine, trietylenephosphoramide, triethylenethiophosphaoramide, trimethylolomelamine, trofosfamide, uracil mustard and uredopa. Suitable anti-metabolites include, but are not limited to aminopterin, ancitabine, azacitidine, 6-azauridine, capecitabine, carmofur (1-hexylcarbomoyl-5-fluorouracil), cladribine, cytarabine or cytosine arabinoside (Ara-C), dideoxyuridine, denopterin, doxifluridine, enocitabine, floxuridine, fludarabine, 5-fluorouracil, gemcetabine, hydroxyurea, leucovorin (folinic acid), 6-mercaptopurine, methotrexate, pemetrexed, pteropterin, thiamiprine, trimetrexate, and thioguanine. Non-limiting examples of suitable anti-tumor antibiotics include aclacinomysin, actinomycins, adriamycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, streptonigrin, streptozocin, tubercidin, valrubicin, ubenimex, zinostatin, and zorubicin. Non-limiting examples of suitable anti-cytoskeletal agents include colchicines, docetaxel, macromycin, paclitaxel, vinblastine, vincristine, vindesine, and vinorelbine. Suitable topoisomerase inhibitors include, but are not limited to, amsacrine, etoposide (VP-16), irinotecan, mitoxantrone, RFS 2000, teniposide, and topotecan. Non-limiting examples of suitable anti-hormonal agents such as aminoglutethimide, aromatase inhibiting 4(5)-imidazoles, bicalutamide, finasteride, flutamide, goserelin, 4-hydroxytamoxifen, keoxifene, leuprolide, LY117018, mitotane, nilutamide, onapristone, raloxifene, tamoxifen, toremifene, and trilostane. Examples of targeted therapeutic agents include, without limit, monoclonal antibodies such as alemtuzumab, epratuzumab, gemtuzumab, ibritumomab tiuxetan, rituximab, tositumomab, and trastuzumab; protein kinase inhibitors such as bevacizumab, cetuximab, dasatinib, erlotinib, gefitinib, imatinib, lapatinib, mubritinib, nilotinib, panitumumab, pazopanib, sorafenib, sunitinib, and vandetanib; angiogeneisis inhibitors such as angiostatin, endostatin, bevacizumab, genistein, interferon alpha, interleukin-2, interleukin-12, pazopanib, pegaptanib, ranibizumab, rapamycin, thalidomide; and growth inhibitory polypeptides such as erythropoietin, interleukins (e.g., IL-1, IL-2, IL-3, IL-6), leukemia inhibitory factor, interferons, thrombopoietin, TNF-α, CD30 ligand, 4-1 BB ligand, and Apo-1 ligand. Also included are pharmaceutically acceptable salts, acids, or derivatives of any of the above listed agents. The mode of administration of the chemotherapeutic agent can and will vary depending upon the agent and the type of cancer. A skilled practitioner will be able to determine the appropriate dose of the chemotherapeutic agent.

The radiotherapeutic agent may include a radioisotope. Suitable radioisotopes include, without limit, Iodine-131, Iodine-125, Iodine-124, Lutecium-177, Phosphorous-132, Rhenium-186, Strontium-89, Yttrium-90, Iridium-192, and Samarium-153. Alternatively, the radiotherapeutic agent may include a high Z-element chosen from gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium. The appropriate dose of the radiotherapeutic agent may be determined by a skilled practitioner.

### (VI) Methods for Preparing Compounds Comprising Formulas (II) or (III)

Yet another aspect of the present disclosure encompasses methods for preparing the compounds comprising Formulas (II) or (III). Those skilled in the art recognize that the disclosed compounds may be prepared by a variety of techniques, including those detailed below.

### (a) Compounds Comprising Formula (IIIa)

Compounds comprising Formula (IIIa) may be prepared by Michael addition reactions. In particular, the method comprises contacting an appropriate parthenolide with the appropriate amine at room temperature or reflux temperature of a suitable solvent (Janganati et al., 2014, Bioorg. Med. Chem. Lett. 24:1963-1967). In general, the mole to mole ratio of the parthenolide compound to the amine may range from about 1:3 to 3:1, e.g., the ratio may be about 1:3, 1:2. 1:1, 2:1, or 3:1. In specific embodiments, the mole to mole ratio of the parthenolide compound to the amine may be about 1:1.

The identity of the solvent may vary depending upon the identity of the parthenolide and the amine. In some embodiments, the solvent may be a protic polar solvent such as methanol, ethanol, isopropanol, water, or propylene glycol. In other embodiments, the solvent may be an aprotic polar solvent such as acetone, acetonitrile, diethoxymethane, N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMAC), N-methyl-2-pyrrolidinone (NMP), 1,4-dioxane, ethyl acetate, ethyl formate, formamide, hexachloroacetone, hexamethylphosphoramide, methyl acetate, N-methylacetamide, methylethyl ketone, methylisobutyl ketone, N-methylformamide, methylene chloride, methoxyethane, morpholine, nitrobenzene, nitromethane, propionitrile, propyl acetates, sulfolane, tetramethylurea, tetrahydrofuran (THF), 2-methyl tetrahydrofuran, tetrahydropyran, trichloromethane, and combinations thereof. In further embodiments, the solvent may be a nonpolar solvent such as benzene, butyl acetate, tert-butyl methyl ether, chlorobenzene, chloroform, chloromethane, cyclohexane, dichloromethane, dichloroethane, di-tert-butyl ether, dimethyl ether, diethylene glycol, diethyl ether, diglyme, diisopropyl ether, ethyl tert-butyl ether, ethylene oxide, fluorobenzene, heptane, hexane, methyl tert-butyl ether, toluene, and combinations thereof. In specific embodiments, the solvent may be methanol or chloroform.

Typically, the reaction is conducted at room temperature and is allowed to proceed until the reaction is complete, as monitored by suitable means such as TLC or HPLC, for example. In various embodiments, the reaction may be allowed to proceed for about 12 hours, about 15 hours, about 18 hours, or about 24 hours. The reaction product may be isolated by means well known in the art. Suitable means include extracting, washing, precipitating, filtering, distilling, and/or chromatography. The yield of the reaction product generally is at least about 50%, or at least about 70%.

### (b) Compounds Comprising Formula (IIIb)

Compounds comprising Formula (IIIb) may be prepared by E-olefinic coupling of parthenolide with an appropriate halo-(hetero)aromatic compound in the presence of a proton acceptor and a palladium catalyst (Han et al., 2009, J. Org. Chen. 74:7176-7179). The mole to mole ratio of the parthenolide compound to the halo-(hetero)aromatic compound may range from about 1:3 to 3:1, e.g., the ratio may be about 1:3, 1:2. 1:1, 2:1, or 3:1. In specific embodiments, the mole to mole ratio of the parthenolide compound to the halo-(hetero)aromatic compound may be about 1:1.

A variety of proton acceptors may be used in the reaction. Suitable proton acceptors include borate salts (such as, for example, NaBO₃), di- and tri-basic phosphate salts (such as, for example, Na₂HPO₄ and Na₃PO₄, and the like), bicarbonate salts (such as, for example, NaHCO₃, KHCO₃, LiCO₃, and the like), carbonate salts (such as, for example, Na₂CO₃, K₂CO₃, Li₂CO₃, and the like), organic bases (such as, for example, pyridine, triethylamine, diisopropylethylamine, N-methylmorpholine, N,N-dimethylaminopyridine), and mixtures of any of the above. In certain embodiments, the proton acceptor is triethylamine. The mole to mole ratio of the proton acceptor to the parthenolide may range from about 1:1 to about 10:1. In specific embodiments, the mole to mole ratio of the proton acceptor to the parthenolide may be about 4:1, about 3:1, or about 2:1.

The palladium catalyst used in the reaction may comprise a variety of forms. In some embodiments, the palladium catalyst may be a sponge or powder, such as palladium powder, palladium sponge, or palladium black. In other embodiments, the palladium may be immobilized on a solid surface or support, such as palladium on carbon, palladium on alumina, palladium on silica, etc. In still further embodiments, the palladium catalyst may be a palladium salt. Non-limiting examples of suitable salts include acetates, acetyacetonates, alkoxides, butyrates, carbonyls, dioxides, halides, hexonates, hydrides, mesylates, octanates, nitrates, nitrosyl halides, nitrosyl nitrates, sulfates, sulfides, sulfonates, phosphates, trifluoromethanesulfonates, tri methylacetates, tosylates, and combinations thereof. The palladium salt may be soluble (i.e., homogeneous). Alternatively, the palladium salt may be immobilized on a solid support (i.e., heterogeneous) via noncovalent or covalent bonds. In some embodiments, the solid support may be an inorganic material. Suitable inorganic materials include silicas, alumina, titania, carbondium, zirconia, activated charcoal, zeolites, clays, polymers, ceramics, and activated carbon. Suitable silicas include silicon dioxide, amorphous silica, and microporous or mesoporous silicas. In other embodiments, the solid support may be a polymer. The polymer may be a natural polymer, a synthetic polymer, a semi-synthetic polymer, or a copolymer. Non-limiting examples of polymers include agarose, cellulose, nitrocellulose, methyl cellulose, polyacrylic, polyacrylamide, polyacrylonitrile, polyamide, polyether, polyester, polyethylene, polystyrene, polysulfone, polyvinyl chloride, polyvinylidene, methacrylate copolymer, and polystyrene-vinyl chloride copolymer.

In further embodiments, the palladium catalyst may be a palladium metal complex comprising palladium and coordinate species with oxidation states ranging from 0 to 8. The complexes may be ionic, or the complexes may comprise covalently bound ligands and counter ions. Alternatively, the complexes may comprise a mixture of ionic and covalent bonds between the metal, ligand(s), and/or counter ion(s). The ligand may be monodentate or polydentate. Non-limiting examples of suitable ligands include arene ligands, olefin ligands, alkyne ligands, heterocycloalkyl ligands, heteroaryl ligands, alkyl ligands, cyclopentadienyl ligands, hydride ligands, amine ligands, carbonyl ligands, nitrogen donor ligands, phosphorous donor ligands, oxygen donor ligands, and so forth. The ligand may also be a solvent such as, e.g., dichloromethane, DMSO, methanol, methylene chloride, tetrahydrofuran, acetone, ethanol, pyridine, or a tetraalkylammonia compound. Suitable counter ions include, but are not limited to, halides, BF₄, PF₆, ClO₄, CHO₂, CF₃SO₃, CH₃CO₂, ArCO₂, CH₃SO₃, p-tolylSO₃, HSO₄, H₂PO₄, and hydrocarbyl anions.

In specific embodiments, exemplary palladium catalysts include Pd(OAc)₂, Pd(dba)₂, Pd(dppf)Cl₂, Pd(acac)₂, [Pd(allyl)Cl]₂, Pd(MeCN)₂Cl₂, Pd(TFA)₂, Pd₂(dba)₃.CHCl₃, Pd(PPh₃)₄, Pd(PCy₃)₂Cl₂, Pd(PPh₃)₂Cl₂, Pd[P(o-tol)₃]₂Cl₂, Pd(amphos)Cl₂, Pd(dtpf)Cl₂, Pd(MeCN)₄(BF₄)₂, PdBr₂, PdCl₂, (SPhos) Pd(II) phenethylamine chloride, (XPhos) Pd(II) phenethylamine chloride, (RuPhos) Pd(II) phenethylamine chloride, (t-BuXPhos) Pd(II) phenethylamine chloride, and (BrettPhos) Pd(II) phenethylamine chloride.

The mole to mole ratio of the palladium catalyst to the parthenolide may range from about 0.001:1 to about 0.1:1. In some embodiments, the mole to mole ratio of the palladium catalyst to the parthenolide may range from about 0.001-0.003 to 1, from about 0.003-0.01 to 1, from about 0.01-0.3 to 1, or from about 0.3-0.1 to 1. In certain embodiments, the mole to mole ratio of the palladium catalyst to the parthenolide may be about 0.01:1.

Suitable solvents are listed above. In specific embodiments, the solvent may be DMF. The temperature of the reaction may range from about 30-50° C., 50-70° C., from about 70-90° C., from about 90-120° C., or from abut 120-160° C. The duration of the reaction may range as detailed above, and the reaction product may be isolated as detailed above. The yield of the reaction product typically is at least about 50%, or at least about 70%.

### (c) Compounds Comprising Formula (II)

Parthenolide derivatives, i.e., comprising Formulas (IIIa) or (IIIb), may be converted into the corresponding melampomagnolide B analogs, i.e., the compounds comprising Formulas (IIa) or (IIb), by utilizing seleniumd dioxide and t-butyl hydroperoxide reagents (Nasim et al., 2011, Bioorg. & Med. Chem. 19: 1515-1519).

### DEFINITIONS

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The compounds described herein can exist in tautomeric, geometric or stereoisomeric forms. The present disclosure contemplates all such compounds, including cis- and trans-geometric isomers, E- and Z-geometric isomers, R- and S-enantiomers, diastereomers, d-isomers, l-isomers, the racemic mixtures thereof, and other mixtures thereof. Pharmaceutically acceptable salts of such tautomeric, geometric or stereoisomeric forms are also included within the invention. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. The terms “cis” and “trans” (or “E” and “Z”), as used herein, denote a form of geometric isomerism in which two carbon atoms connected by a double bond will each have a hydrogen atom on the same side of the double bond (“cis”) or on opposite sides of the double bond (“trans”).

The term “acyl,” as used herein alone or as part of another group, denotes the moiety formed by removal of the hydroxyl group from the group COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkoxide” or “alkoxy” as used herein is the conjugate base of an alcohol. The alcohol may be straight chain, branched, cyclic, and includes aryloxy compounds.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “protecting group” as used herein denotes a group capable of protecting a particular moiety, wherein the protecting group may be removed, subsequent to the reaction for which the protection is employed, without disturbing the remainder of the molecule. A variety of protecting groups and the synthesis thereof may be found in “Greene's Protective Groups in Organic Synthesis,” 4^{th} Ed. by P. G. M. Wuts and T. W. Greene, John Wiley & Sons, Inc., 2007.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

Having described the invention in detail, it will be apparent that modifications and variations are possible without departing from the scope of the invention defined in the appended claims.

### Examples

### Example 1

### Synthesis of 13-(2-aminobenzimidazole)parthenolide (JVM-9)

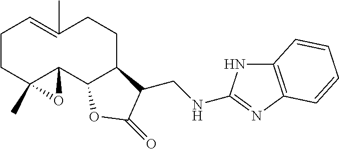

To a stirred solution of parthenolide (50 mg, 0.201 mmol) in methanol, aminobenzimidazole (26.84 mg, 0.201 mmol) was added. The reaction mixture was stirred at ambient temperature for 15 h. After completion of the reaction (monitored by TLC), the reaction mixture was concentrated under reduced pressure to afford the crude compound, and the crude compound was purified by column chromatography (dichloromethane/methanol; 92:8) to afford the final compound as white solid (yield: 85%). MP 152° C. ¹H NMR (CDCl₃, 400 MHz): δ 7.42 (d, J=8 Hz, 1H), 7.137 (t, J=8 Hz, 1H), 7.05 (t, J=8 Hz 1H), 6.99 (d, J=8 Hz, 1H), 5.46 (brs, 1H), 5.02 (d, J=12 Hz, 1H), 4.37 (ddd, J=16, 4 Hz, 2H), 3.88 (t, J=8 Hz, 1H), 2.79 (d, J=12 Hz, 1H), 2.59 (d, J=12 Hz, 1H), 2.36-2.27 (m, 2H), 2.17-1.93 (m, 6H), 1.79-1.70 (m, 1H), 1.65 (s, 3H), 1.24 (s, 3H), 1.34-1.07 (m, 1H). ¹³C NMR (100 MHz, CDCl₃): δ 176.7, 154.6, 141.8, 134.7, 133.9, 125.9, 122.4, 120.1, 116.8, 107.2, 83.3, 65.6, 61.9, 48.8, 46.7, 40.7, 40.4, 36.4, 30.1, 24.1, 17.2, 16.9 ppm.

### Example 2

### Synthesis of 13-(aminoethylmorpholino)parthenolide (JVM-11)

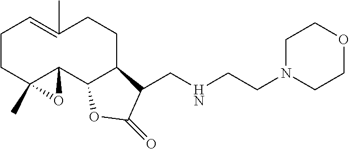

To a stirred solution of parthenolide (50 mg, 0.201 mmol) in chloroform, aminoethylmorpholine (26.13 mg, 0.201 mmol) was added. The reaction mixture was stirred at ambient temperature for 15 h. After completion of the reaction (monitored by TLC), the reaction mixture was concentrated under reduced pressure and the crude compound was purified by column chromatography (dichloromethane/methanol; 91:9) to afford the final compound as a colorless oil (yield 85%). ¹H NMR (CDCl₃, 400 MHz): δ 5.17 (d, J=12 Hz, 1H), 3.82 (t, J=8 Hz, 1H), 3.69 (t, J=4 Hz, 4H), 3.69 (dd, J=12, 4 Hz, 1H), 2.82-2.69 (m, 4H), 2.49-2.02 (m, 15H), 1.99-1.89 (m, 1H), 1.67 (s, 3H), 1.26 (s, 3H), 1.19-1.18 (m, 1H). ¹³C NMR (100 MHz, CDCl₃): δ 176.6, 134.4, 125.1, 82.5, 66.9, 66.2, 61.4, 57.9, 53.6, 47.7, 47.5, 46.8, 46.4, 41.0, 36.5, 30.0, 24.0, 17.1, 16.8 ppm.

### Example 3

### Synthesis of 13-(2-iodoimidazole)parthenolide (PNR-5-65)

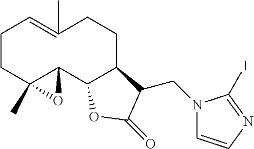

A mixture of parthenolide (50 mg, 0.2 mmol) and 2-iodoimidazole (43 mg, 0.22 mmol), was stirred at reflux temperature for 24 h in methanol (10 ml). The reaction mixture was cooled to room temperature, concentrated under reduced pressure and the residue was purified by using silica flash chromatography (1% to 5%, methanol in dichloromethane) to afford 2-iodoimidazole parthenolide as a white solid (60 mg) in 67 yield: MP 199-200° C.; ¹H NMR (DMSO-d₆, 400 MHz): δ 7.34 (s, 1H), 6.99 (s, 1H), 5.11-5.14 (d, J=10 Hz, 1H), 4.20-4.30 (ddd, J=9.2, 5.6 Hz, 2H), 4.03-4.07 (t, J=9.2 Hz, 1H), 3.17 (m, 1H), 2.99-3.04 (m, 1H), 2.69-2.71 (d, J=8.8 Hz, 1H), 2.30-2.34 (s, 1H), 194-2.14 (m, 5H), 1.59 (s, 3H), 1.28-1.31 (m, 1H), 1.17 (s, 3H), 1.06-1.20 (m, 1H); ¹³C NMR (100 MHz, CDCl₃) δ 174.94, 134.18, 131.95, 124.37, 124.07, 93.23, 81.54, 65.15, 61.15, 47.20, 46.16, 40.31, 35.85, 28.68, 23.51, 16.68, 16.48 ppm.

### Example 4

### Synthesis of 13-(4-aminophenyl)parthenolide (PNR-5-41)

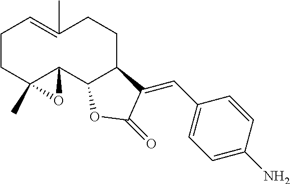 [SMILES: C/C1=C\CC[C@@]2(C)O[C@H]2[C@H]2OC(=O)/C(=C\c3ccc(N)cc3)[C@@H]2CC1]

A mixture of parthenolide (50 mg, 0.20 mmol), triethylamine (60 mg, 0.61 mmol), and 4-iodoaniline (48.56 mg, 0.22 mmol) in DMF (0.1 ml) was treated with palladium(II) acetate (0.5 mg, 0.002 mmol) and then heated at 80° C. under air. After 24 h, the reaction mixture was allowed to cool to room temperature, water (8 mL) was added, and the resultant mixture was extracted with ethyl acetate (10 ml×3). The separated organics were dried over Na₂SO₄ and concentrated under reduced pressure. The obtained crude residue was purified by using silica flash chromatography. (hexanes/EtOAc; 9:1-4:1) to afford the 4-amino phenyl parthenolide as a white solid (40 mg) in 58% yield: MP 239-241° C.; ¹H NMR (400 MHz, CDCl₃): δ 7.54-7.55 (d, J=2.8 Hz, 1H), 7.23-7.26 (d, J=8.4 Hz, 2H), 6.68-6.70 (d, J=8.0 Hz, 2H), 5.28-5.30 (d, J=11.2 Hz, 1H), 4.02 (brs, 2H), 3.91-3.95 (t, J=8.0 Hz, 1H), 3.25 (m, 1H), 2.82-2.84 (d, J=8.8 Hz, 1H), 2.28-2.45 (m, 1H), 2.14-2.24 (m, 5H), 1.69 (s, 3H), 1.61 (s, 1H), 1.43-1.45 (m, 1H), 1.31 (s, 3H); ¹³C NMR (100 MHz, CDCl₃) δ 171.63, 148.15, 138.64, 134.79, 132.17, 125.12, 124.03, 123.29, 114.42, 82.71, 66.62, 61.58, 46.98, 41.88, 36.10, 29.33, 24.33, 17.49, 17.36 ppm.

### Example 5

### Synthesis of (E)-13-(3-thiopheno)parthenolide (PNR-5-53)

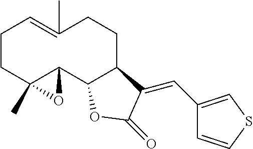 [SMILES: C/C1=C\CC[C@@]2(C)O[C@H]2[C@H]2OC(=O)/C(=C\c3ccsc3)[C@@H]2CC1]

A mixture of parthenolide (50 mg, 0.20 mmol), triethylamine (60 mg, 0.61 mmol), and 3-iodothiophene (46 mg, 0.21 mmol) in DMF (0.1 ml) was treated with palladium(II) acetate (0.5 mg, 0.002 mmol) and then heated at 80° C. under air. After 24 h, the reaction mixture was allowed to cool to room temperature, water (8 mL) was added, and the resultant mixture was extracted with ethyl acetate (10 ml×3). The separated organics were dried over Na₂SO₄ and concentrated under reduced pressure. The obtained crude residue was purified by using silica flash chromatography (hexanes/EtOAc; 9:1-4:1) to afford the (E)-13-(3-thiopheno)parthenolide as a white solid (35 mg) in 52% yield. MP 183-185° C.; ¹H NMR (400 MHz, CDCl₃): δ 7.65 (d, J=2.8 Hz, 1H), 7.51 (s, 1H), 7.41 (s, 1H), 7.20-7.21 (d, J. 4.4 Hz, 1H), 5.28-5.31 (d, J=12 Hz, 1H), 3.96-4.00 (t, J=6.8 Hz, 1H), 3.18 (m, 1H), 2.82-2.84 (d, J=8.8 Hz, 1H), 2.43 (m, 1H), 2.16-2.30 (m, 5H), 1.72 (s, 3H), 1.54 (m, 1H), 1.33 (s, 3H), 1.26-1.31 (m, 1H); ¹³C NMR (100 MHz, CDCl₃) δ 171.09, 135.37, 134.52, 131.85, 129.28, 127.78, 127.33, 126.49, 125.42, 82.7, 66.41, 61.64, 46.92, 42.10, 35.99, 30.56, 24.36, 17.52, 17.30 ppm.

### Example 6

### Synthesis of (E)-13-(Phenyl)Melampomagnolide B (PNR-5-38)

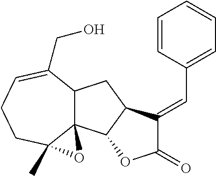 [SMILES: C[C@@]12CCC=C(CO)C3C[C@H]4/C(=C\c5ccccc5)C(=O)O[C@@H]4[C@@]31O2]

A mixture of (E)-13-(phenyl)parthenolide (50 mg, 0.15 mmol), selenium dioxide (8.5 mg, 0.76 mmol), t-butyl hydroperoxide (54 mg, 0.6 mmol) were stirred in dichloromethane for 16 hrs. Concentrated the reaction mass and the residue obtained was purified by using silica gel column chromatography. (hexanes/EtOAc; 9:1-4:1) to afford the (E)-13-(phenyl) melampomagnolide B as a white solid (35 mg) in 67% yield. MP 115-117° C.; ¹H NMR (400 MHz, CDCl₃): δ 7.73 (s, 1H), 7.35-7.43 (m, 5H), 5.63-5.67 (t, J=16 Hz, 7.6 Hz, 1H), 3.83-3.88 (t, J=17.6, Hz, 8.4 Hz, 1H), 3.24-3.29 (dd, 1H), 2.90-2.92 (d, J=8.8 Hz, 1H), 2.34-2.38 (m, 2H), 2.10-2.24 (m, 5H), 1.54-1.67 (m, 2H), 1.52 (s, 3H), 1.21-1.1.25 (m, 2H); ¹³C NMR (100 MHz, CDCl₃) δ 171.01, 139.74, 138.95, 134.19, 129.69, 129.28, 128.76, 128.42, 127.16, 80.49, 65.71, 63.06, 60.5, 60.37, 42.75, 36.50, 24.53, 24.41, 23.62, 18.14, 14.16 ppm.

### Example 7

### In Vitro Growth Inhibition and Cytotoxicity

In a primary screen, all these synthesized compounds were evaluated at 10 μM concentration for their cytotoxic potency at the National Cancer Institute (NCI). The compounds were considered to be active if they reduced the growth of any of the cancer cell lines 60% or more in at least eight of the cell lines screened. If this criterion was met, the compounds were then passed on for evaluation in a full panel of 60 different cancer cell lines. From the preliminary 60 cell line screen, the compounds which showed ≧60% growth inhibition in at least eight of the cancer cell lines screened were selected for further five dose studies. From all these compounds, the two most active compounds (PNR-5-53, PNR-5-65) were subsequently evaluated in five dose-response studies for their in vitro cytotoxic effects on growth parameters against each of the 60 human tumor cell lines. The compound with thiophene-3-yl moiety (PNR-5-53) exhibited growth inhibitory properties against all cancer cell lines in the panel. Growth inhibition is presented as GI₅₀ (50% growth inhibition, concentration of drug resulting in a 50% reduction in net cell growth as compared to cell numbers on day 0). The GI₅₀ values of PNR-5-53 range from 5.44 to 48.1 μM (Table 1), with good growth inhibitory activity against NCI-H522 non-small cell lung cancer cells (GI₅₀=5.44 μM; LC₅₀=59.7 μM), leukemia CCRF-CEM (GI₅₀=19.1 μM; LC₅₀=>100 μM) and RPMI-8226 (GI₅₀=19.7 μM; LC₅₀=>100 μM) cancer cells lines and CNS cancer SF-539 (GI₅₀=16.1 μM; LC₅₀=>62.2 μM), melanoma UACC-62 (GI₅₀=13.8 μM; LC₅₀=76.6 μM), SK-MEL-5 (GI₅₀=16.3 μM; LC₅₀=60.4 μM), renal A498 (GI₅₀=17.5 μM; LC₅₀=>100 μM), breast cancer MDA-MB-231/ATCC (GI₅₀=16.0 μM; LC₅₀=81.8 μM) cancer cell lines.

The compound with 2-imidazole moiety (PNR-5-65) exhibited growth inhibitory properties against all the cancer cell lines in the panel, with GI₅₀ values in the range of 1.71 to 47.4 μM except SNB-19 CNS cancer cell lines (Table 1). The compound PNR-5-65 exhibited potent growth inhibitory activity against NCI-H522 non-small cell lung cancer cells (GI₅₀=1.77 μM; LC₅₀=6.34 μM), and shows good growth inhibition on leukemia CCRF-CEM (GI₅₀=6.13 μM; LC₅₀=79.6 μM), and HL-60(TB) (GI₅₀=6.90 μM; LC₅₀=77.5 μM), cancer cell lines, colon HCT-15 (GI₅₀=10.7 μM; LC₅₀=55.6 μM), melanoma MALME-3M (GI₅₀10.9 μM; LC₅₀=62.7 μM), ovarian OVCAR-3 (GI₅₀=6.85 μM; LC₅₀=50.8 μM), renal CAKI-1 (GI₅₀=6.61 μM; LC₅₀=49.5 μM), ACHN (GI₅₀=10.5 μM; LC₅₀=47.4 μM), UO-31 (GI₅₀10.8 μM; LC₅₀=50 μM), breast cancer MCF7 (GI₅₀=9.79 μM; LC₅₀=>100 μM), T-47D (GI₅₀=9.88 μM; LC₅₀=54.3 μM) cell lines.

TABLE 1
Antitumor activity data of the compounds selected for 5 dose studies
for the NCI 60-cell lines screen

| | |
|---|---|
| PNR-5-53 | PNR-5-65 |

| | | | | |
|---|---|---|---|---|
| Panel/cell line | GI₅₀ | LC₅₀ | GI₅₀ | LC₅₀ |

Leukemia

| | | | | |
|---|---|---|---|---|
| CCRF-CEM | 19.1 | >100 | 6.13 | 79.6 |
| HL-60(TB) | 21.7 | 99.9 | 6.90 | 77.5 |
| K-562 | 25.2 | >100 | 7.36 | 90.0 |
| MOLT-4 | 25.7 | >100 | 19.6 | 97.9 |
| RPMI-8226 | 19.7 | >100 | 17.0 | >100 |
| SR | 20.7 | >100 | 13.6 | >100 |

Lung Cancer

| | | | | |
|---|---|---|---|---|
| A549/ATCC | | | | |
| HOP-62 | 35.8 | >100 | 47.4 | >100 |
| NCI-H226 | 30.2 | >100 | 15.4 | 62.3 |
| NCI-H23 | 23.0 | >100 | 23.7 | >100 |
| NCI-H322M | 44.7 | >100 | 61.3 | >100 |
| NCI-H460 | 29.3 | >100 | 30.1 | >100 |
| NCI-H522 | 5.44 | 59.7 | 1.71 | 6.34 |

Colon Cancer

| | | | | |
|---|---|---|---|---|
| COLO 205 | | | | |
| HCC-2998 | 33.8 | >100 | NA* | NA |
| HCT-116 | 28.3 | >100 | 11.8 | 49.0 |
| HCT-15 | 22.6 | >100 | 10.7 | 55.6 |
| HT29 | 27.0 | >100 | 17.6 | 61.5 |
| KM12 | 20.4 | 86.7 | 43.2 | >100 |
| SW-620 | 22.9 | >100 | 11.4 | 62.8 |

CNS Cancer

| | | | | |
|---|---|---|---|---|
| SF-268 | | | | |
| SF-295 | 28.5 | >100 | 32.1 | >100 |
| SF-539 | 16.1 | 62.2 | 16.7 | 60.8 |
| SNB-19 | 47.6 | >100 | >100 | >100 |
| SNB-75 | 21.5 | >100 | 19.3 | >100 |
| U251 | 21.7 | >100 | 36.7 | >100 |

Melanoma

| | | | | |
|---|---|---|---|---|
| LOX IMVI | | | | |
| MALME-3M | 22.5 | >100 | 10.9 | 62.7 |
| M14 | 20.5 | >100 | 16.5 | 65.6 |
| MDA-MB-435 | 18.3 | >100 | 13.8 | 59.5 |
| SK-MEL-2 | NA | NA | 18.6 | 66.1 |
| SK-MEL-28 | 29.4 | >100 | 16.1 | 73.0 |
| SK-MEL-5 | 16.3 | 60.4 | 18.5 | 95.6 |
| UACC-257 | 16.6 | >100 | 12.0 | 53.9 |
| UACC-62 | 13.8 | 76.6 | NA | NA |

Ovarian Cancer

| | | | | |
|---|---|---|---|---|
| IGROV1 | | | | |
| OVCAR-3 | 20.5 | >100 | 6.85 | 50.8 |
| OVCAR-4 | 24.3 | >100 | 13.2 | 60.0 |
| OVCAR-5 | 48.1 | >100 | 13.7 | 52.5 |
| OVCAR-8 | 26.9 | >100 | 16.6 | 59.9 |
| NCI/ADR-RES | 23.6 | >100 | 31.2 | >100 |
| SK-OV-3 | 44.9 | >100 | 47.3 | >100 |

Renal Cancer

| | | | | |
|---|---|---|---|---|
| 786-0 | | | | |
| A498 | 17.5 | >100 | 45.2 | >100 |
| ACHN | 32.8 | >100 | 10.5 | 47.4 |
| CAKI-1 | 30.9 | >100 | 6.61 | 49.5 |
| RXF 393 | 18.1 | >100 | NA | NA |
| SN12C | 25.8 | >100 | 17.7 | 84.1 |
| TK-10 | 34.7 | >100 | NA | NA |
| UO-31 | 27.3 | >100 | 10.8 | 50.0 |

Prostate Cancer

| | | | | |
|---|---|---|---|---|
| PC-3 | 22.7 | >100 | 23.1 | >100 |
| DU-145 | 25.7 | >100 | 18.8 | 68.3 |

Breast Cancer

| | | | | |
|---|---|---|---|---|
| MCF7 | 20.0 | >100 | 9.79 | >100 |
| MDA-MB-231/ATCC | 16.0 | 81.8 | 16.4 | 59.6 |
| HS 578T | 21.5 | >100 | 21.3 | >100 |
| BT-549 | 20.1 | 88.2 | 15.9 | 55.3 |
| T-47D | 29.5 | >100 | 9.88 | 54.3 |
| MDA-MB-468 | 17.7 | >100 | 11.1 | 60.4 |

*NA = Not analyzed.

### Example 8

### Screening Using a Xenopus Oocyte Maturation Assay

An in vivo Xenopus oocyte maturation assay was used to screen the compounds for their ability to modulate the activity of cell cycle control proteins, mRNA translational control proteins, and/or proteins involved in signaling pathways. In this assay, oocyte maturation (or cell cycle progression) is monitored phenotypically using an image capture system. Oocyte maturation is correlated with the appearance of a white spot at the animal pole of the darkly pigmented animal hemisphere of the oocyte. The white spot appears because the germinal vesicle (i.e., the large nucleus of the oocyte) migrates to the animal pole prior to meiotic germinal vesicle breakdown (GVBD).

Immature stage VI Xenopus oocytes were transferred to wells of a flat bottom 96-well plate containing about 178 μL of L15 culture media. The oocytes were transferred using wide mouth 200 μL pipette tips with minimal transfer of culture medium (e.g., about 20 μL). Generally, the oocytes oriented with the pigmented animal hemisphere facing up due to the higher density of the yolk-containing vegetal hemisphere. Incorrectly orientated oocytes were gently manipulated using a drawn-out capillary tube to rotate them into the correct orientation. It was found that 21 oocytes per well was optimal as they formed a regular 13:7:1 monolayer array that filled the flat bottom of the well and, essentially, locked the oocytes in the correct orientation.

Forty-four test compounds were dissolved in DMSO to make 100× stock solutions. The test compounds were added to the appropriate wells at a final concentration of 100 μM (and 1% DMSO). Control oocytes were exposed to 1% DMSO only (generally the first and last wells of a plate contained the untreated, control oocytes). Sixteen hours later, progesterone was added to each well to stimulate progesterone-dependent oocyte maturation. For this, 2 μL of 1000× stock solution (2 mg/mL in ethanol) was added to the appropriate wells to yield a final concentration of 2 μg/mL. The rate of maturation in the control oocytes (i.e., those in the first and last wells) served as an intra-assay control for time taken for progesterone addition across the multi-well plate.

The 96-well plate was imaged from above (i.e., top-down) using a 36-megapixel Nikon D800 SLR camera with an AF Micro Nikkor 60 mm, f/2.8 lens, mounted on a dedicated copy stand (with adjustable flanking light sources). The plate was imaged prior to and after addition of progesterone (i.e., images were taken at regular intervals over a period of 7 hours). The high resolution images were digitally zoomed after capture to analyze the maturation status of oocytes in individual wells of the plate. Test compounds that slowed the rate of progesterone-dependent GVBD by 20% or more were classified as inhibitors (assessed relative to the time 50% of the population of control, vehicle treated oocytes had undergone GVBD (GVBD50)).

To determine whether the test compounds were able to activate oocyte maturation in a progesterone-independent manner, oocytes were incubated in the presence of the test compound (dissolved in DMSO) or DMSO only. The oocytes were imaged at regular intervals for the evidence of GVBD, or subsets of oocytes were removed at predetermined times and the activation status of key cell cycle control proteins (e.g., pMAP kinase, pCcd2, etc.) was analyzed by Western blotting. For example, activation of pMAP kinase is associated with increased phosphorylation whereas activation of pCdc2 is associated with decreased phosphorylation. Compounds were classified as an activator if they were able to induce GVBD in the absence of added progesterone. Further, compounds were classified as accelerators if they accelerated the rate of progesterone-stimulated GVBD, but were unable to induce GVBD in the absence of progesterone. The criteria for test compounds to be considered accelerators is that they must increase the rate of progesterone-dependent GVBD by 20% or more at one dose (assessed relative to the time 50% of the population of control, vehicle treated oocytes had undergone GVBD (GVBD₅₀)). Table 2 presents results from these in vivo screens.

TABLE 2
Activity of Compounds in Xenopus Screening Bioassay

| | | Xenopus Oocyte Maturation Assay | |
|---|---|---|---|
| | | With | Without |
| Compound | Structure | Progesterone | Progesterone |
| PNR-5-41 | 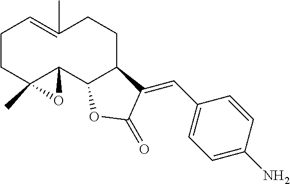 [SMILES: C/C1=C\CC[C@@]2(C)O[C@H]2[C@H]2OC(=O)/C(=C\c3ccc(N)cc3)[C@@H]2CC1] | Inhibitor | |
| PNR-5-65 | 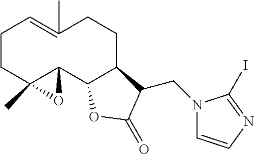 | Not an activator or inhibitor | Not an activator or inhibitor |
| PNR-5-53 | 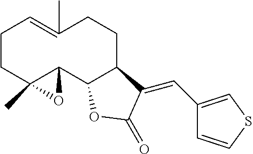 [SMILES: C/C1=C\CC[C@@]2(C)O[C@H]2[C@H]2OC(=O)/C(=C\c3ccsc3)[C@@H]2CC1] | Not an activator or inhibitor | Not an activator or inhibitor |
| JVM-11 | 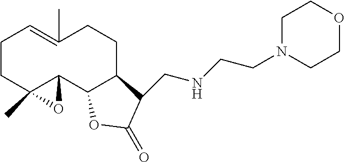 | Inhibitor | |
| PVR-5-46 | 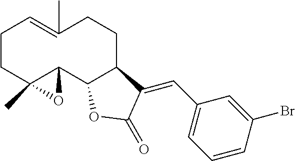 [SMILES: C/C1=C\CC[C@@]2(C)O[C@H]2[C@H]2OC(=O)/C(=C\c3cccc(Br)c3)[C@@H]2CC1] PVR-5-46 | | Activator |
| JVM-9 | 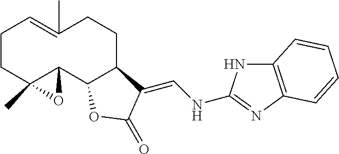 [SMILES: C/C1=C\CC[C@@]2(C)O[C@H]2[C@H]2OC(=O)/C(=C\Nc3nc4ccccc4[nH]3)[C@@H]2CC1] | | Activator |
| JVM-20 | 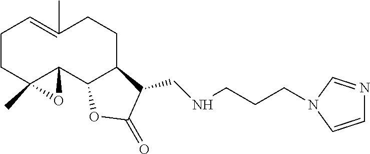 [SMILES: C/C1=C\CC[C@@]2(C)O[C@H]2[C@H]2OC(=O)[C@@H](CNCCCn3ccnc3)[C@@H]2CC1] JVM-20 | Activator (1-50 μM) Inhibitor (100 μM) | |

### Example 9

### Compounds Identified in Xenopus Bioassay Inhibit Mammalian Cancer Cell Self-Renewal

Mammosphere culture growth presents a useful indicator of the presence of breast cancer cells with stem cell-like properties. Breast cancer cell lines grown as mammospheres (under non-adherent plating conditions) recapitulate the three-dimensional organization of tumors. Importantly, assessment of stem cell self-renewal capacity can be achieved through dispersion of mammospheres to single cells and subsequent limited dilution replating. To determine whether PNR-5-46, PNR-5-41, or JVM-20 inhibited mammosphere formation in this system, MCF-7 or MDA MB231 cells were cultured lines for 3 days in media containing the test compound (50 μM) or DMSO, and then cultured in the absence of the test compound. At day 7, mammospheres were collected, dispersed to single cells and replated at limiting dilution (P1). Spheroids were scored at day 14 (P2). Mammosphere forming units (MFUs) are defined as spheroid bodies >100 μm (MCF-7 cells) or >65 μm (MDA MB231 cells) diameter after 7 (P1) or 14 (P2) days growth in non-adherent culture.

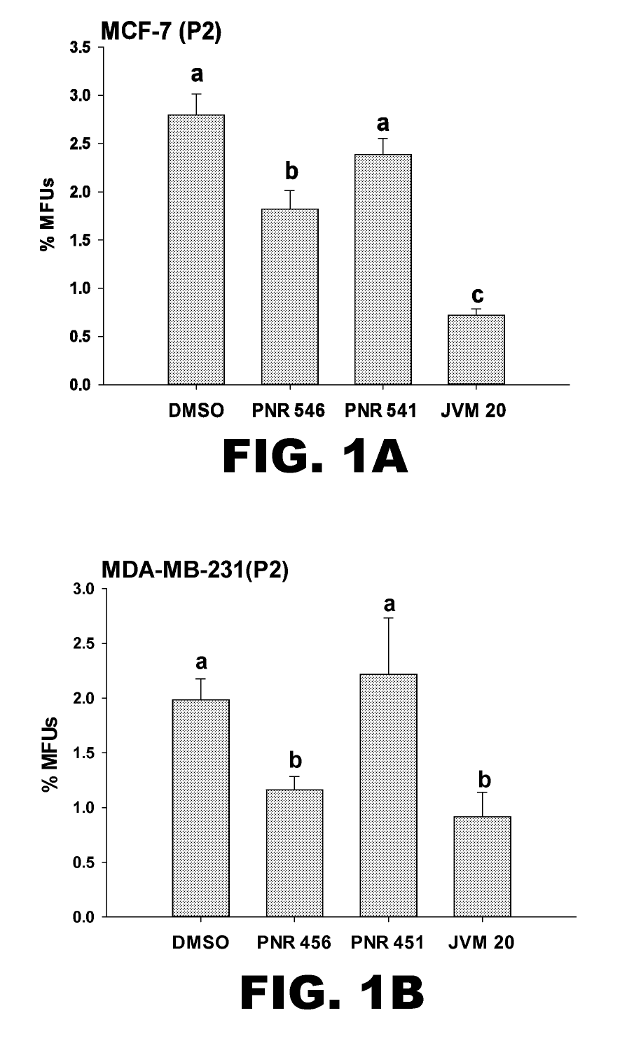

PNR-5-46 and JVM-20 effectively inhibited mammosphere formation of both MCF-7 or MDA MB231 cells relative to DMSO-treated control cells, whereas PNR-5-41 did not (FIG. 1A,B). This effect was observed at 50, 5, and 0.5 μM doses. Since the compounds were only present during the first three days of culture, inhibition of mammosphere formation on day 14 following the dispersion and re-plating on day 7, reflects a sustained impact on the cancer stem cell population. When assessed at the 0.5 μM dose, there was no significant effect of drug treatment on general cell viability, indicating the PNR-5-46 and JVN-20 specifically targeted cancer stem cell functionality. These findings suggest that molecules that impinge upon mRNA translational control during Xenopus oocyte maturation may be active in the regulation of stem cell self-renewal.

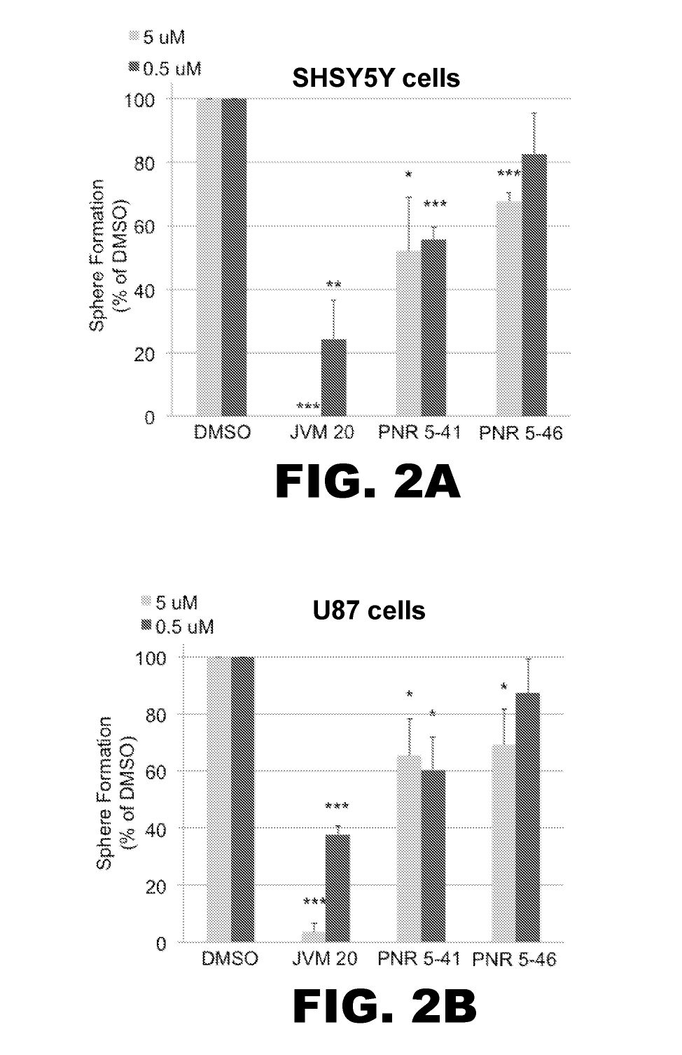

The activity of the three test compounds to inhibit neurosphere formation was also tested in a neuroblastoma cell line SHSY5Y and a glioblastomoa cell line U87. All three compounds attenuated neurosphere formation in both cell lines, to varying degrees of efficacy (FIG. 2A,B). JVM-20 was particularly effective, even at the lowest dose tested (0.5 μM). None of the compounds affected cell viability. Significant inhibition of SHSY5Y and U87 stem cell self-renewal was also seen for PNR-5-41, but not for PNR-5-46. Interestingly, PNR-5-41 was effective for attenuation of neural cancer stem cell function but was not effective against breast cancer stem cells, whereas PNR-5-46 was effective for breast cancer stem cells but was not as effective against neural cancer stem cell at low dose. Together these data indicate a differential sensitivity of breast and neural cancer stem cells to different parthenolide derivatives.

1. A method for modulating a process controlled by regulated mRNA translation, the method comprising contacting a cell with an effective amount of a compound comprising Formula (I) or a pharmaceutically acceptable salt thereof, whereby the process controlled by regulated mRNA translation is attenuated or activated, the compound comprising Formula (I): 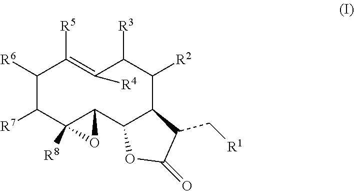
wherein:
R¹ is hydrogen, hydrocarbyl, or substituted hydrocarbyl;
R², R³, R⁶, and R⁷ are independently hydrogen, hydroxyl, keto, halo, amine, amide, nitro, phospho, cyano, thiol, hydrocarbyl, or substituted hydrocarbyl;
R⁴, R⁵, and R⁸ are independently hydrogen, hydroxyl, halo, amine, amide, nitro, phospho, thiol, hydrocarbyl, or substituted hydrocarbyl; and
----- is a double or single bond.

2. The method of claim 1, wherein R¹ is hydrogen, alkyl, substituted alkyl, alkenyl, substituted alkenyl, alkynyl, substituted alkynyl, amine, carbocyclic, substituted carbocyclic, heterocyclic, or substituted heterocyclic; each of R², R³, R⁵, R⁶, and R⁷ is hydrogen; R⁴ is alkyl, alkyl alcohol, ether, or ester; and R⁸ is alkyl.

3. (canceled)

4. The method of claim 1, wherein regulated mRNA translation is regulated by a sequence-specific mRNA binding protein chosen from a Pumilio, a Musashi, or a cytoplasmic polyadenylation element binding (CPEB) protein.

5. The method of claim 1, wherein the process controlled by regulated mRNA translation is cell cycle progression, cell growth control, cell division control, cell survival control, or signal transduction.

6. The method of claim 1, wherein the cell is disposed in a subject.

7. A compound comprising Formula (II) or a pharmaceutically acceptable salt thereof: 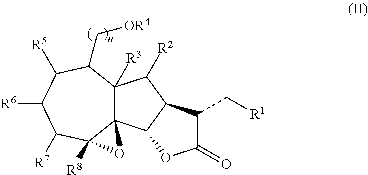
wherein:
R¹ is hydrocarbyl or substituted hydrocarbyl;
R², R³, R⁶, and R⁷ are independently hydrogen, hydroxyl, keto, halo, amine, amide, nitro, phospho, cyano, thiol, hydrocarbyl, or substituted hydrocarbyl;
R⁴ is hydrogen, hydrocarbyl, or substituted hydrocarbyl;
R⁵ and R⁸ are independently hydrogen, hydroxyl, halo, amine, amide, nitro, phospho, thiol, hydrocarbyl, or substituted hydrocarbyl;
n is an integer from 1 to 4; and
----- is a double or single bond.

8. The compound of claim 7, wherein R¹ is alkyl, substituted alkyl, alkenyl, substituted alkenyl, alkynyl, substituted alkynyl, amine, carbocyclic, substituted carbocyclic, heterocyclic, or substituted heterocyclic; each of R², R³, R⁵, R⁶, and R⁷ is hydrogen; R⁴ is hydrogen, alkyl, substituted alkyl, alkenyl, substituted alkenyl, alkynyl, substituted alkynyl, aryl, or substituted aryl; and R⁸ is alkyl.

9. The compound of claim 7, wherein the optional single bond is present and R¹ is —NR⁹R¹⁰, in which R⁹ is hydrogen and R¹⁰ is alkyl, substituted alkyl, alkenyl, substituted alkenyl, alkynyl, substituted alkynyl, carbocyclic, substituted carbocyclic, heterocyclic, or substituted heterocyclic, or together R⁹ and R¹⁰ form a ring or ring system chosen from carbocyclic, substituted carbocyclic, heterocyclic, or substituted heterocyclic.

10. The compound of claim 7, wherein the optional double bond is present and R¹ is carbocyclic, substituted carbocyclic, heterocyclic, or substituted heterocyclic.

11. The compound of claim 7, wherein the compound comprises Formula (IIa): 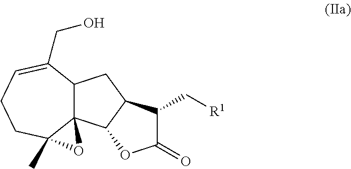
wherein:
R¹ is —NR⁹R¹⁰, in which R⁹ is hydrogen and R¹⁰ is alkyl, substituted alkyl, alkenyl, substituted alkenyl, alkynyl, substituted alkynyl, carbocyclic, substituted carbocyclic, heterocyclic, or substituted heterocyclic, or together R⁹ and R¹⁰ form a ring or ring system chosen from carbocyclic, substituted carbocyclic, heterocyclic, or substituted heterocyclic.

12. The compound of claim 11, wherein R¹ is chosen from:

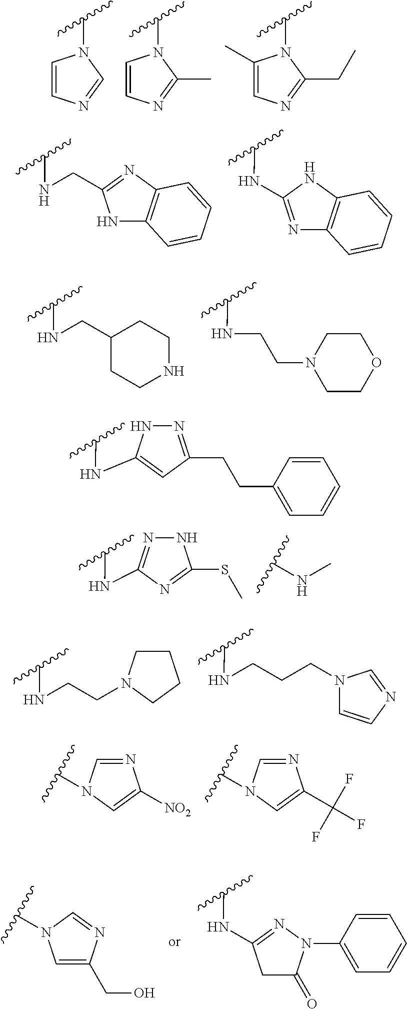

13. The compound of claim 7, wherein the compound comprises Formula (IIb): 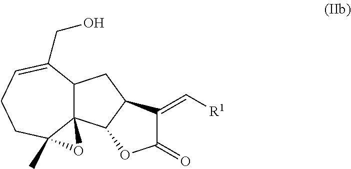
wherein:
R¹ is carbocyclic, substituted carbocyclic, heterocyclic, or substituted heterocyclic.

14. The compound of claim 13, wherein R¹ is chosen from:

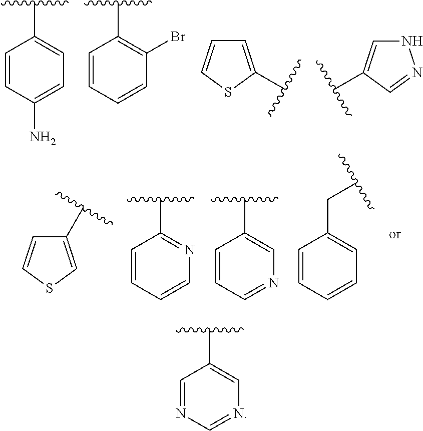

15. A pharmaceutical composition comprising the compound of claim 7 and at least one pharmaceutically acceptable excipient.

16. A compound comprising Formula (III) or a pharmaceutically acceptable salt thereof: 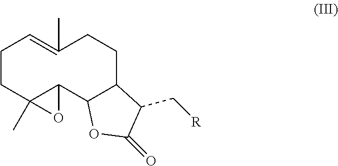
wherein:
----- is a double or single bond; and
R is —NR⁹R¹⁰, in which R⁹ is hydrogen and R¹⁰ is heterocyclic or substituted heterocyclic, or together R⁹ and R¹⁰ form heterocyclic or substituted heterocyclic when ----- is a single bond; or
R is carbocyclic, substituted carbocyclic, heterocyclic, or substituted heterocyclic when ----- is a double bond.

17. The compound of claim 16, wherein the compound comprises Formula (IIIa): 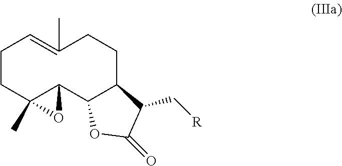
wherein R is chosen from:

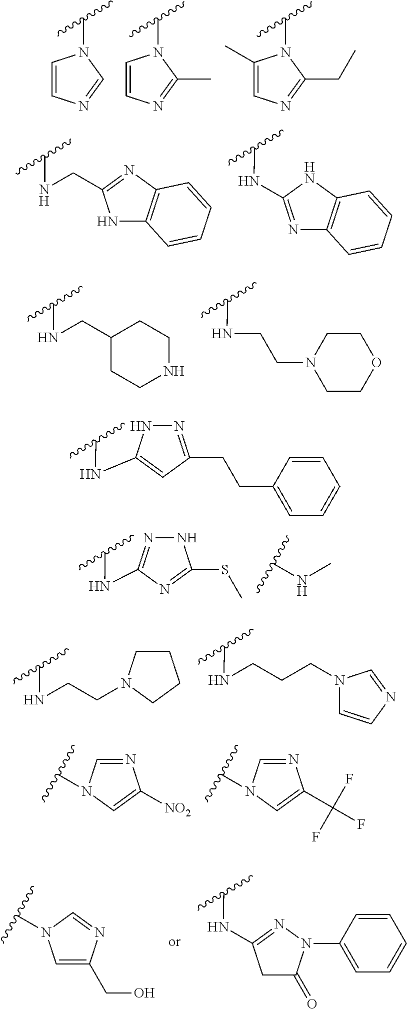

18. The compound of claim 16, wherein the compound comprises Formula (IIIb): 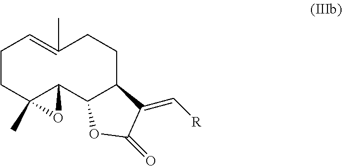
wherein R is chosen from:

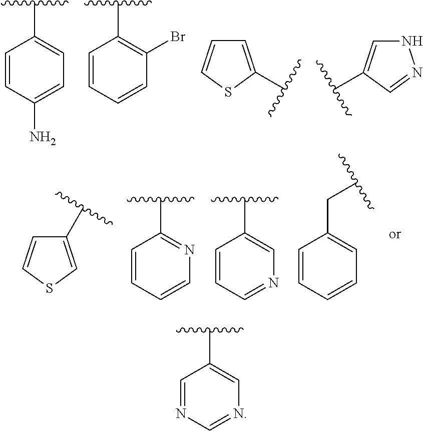

19. A pharmaceutical composition comprising the compound of claim 16 and at least one pharmaceutically acceptable excipient.

20. A method for inhibiting growth of a cancer cell, the method comprising contacting the cancer cell with an effective amount of a compound of claim 7, or a pharmaceutically acceptable salt of either.

21. (canceled)

22. (canceled)

23. (canceled)

24. (canceled)

25. (canceled)

26. A method for inhibiting growth of a cancer cell, the method comprising contacting the cancer cell with an effective amount of a compound of claim 16, or a pharmaceutically acceptable salt of either.
